(19) United States
(12) Patent Application Publication
(10) Pub. No.: US 2026/0284391 A1
(43) Pub. Date: Sep. 24, 2026

(54) OBJECTIVE MEASUREMENTS FOR DETERMINING CHANNEL INTERACTION OF A COCHLEAR IMPLANT

(71) Applicant: MED-EL Elektromedizinische Geräte GmbH, Innsbruck (AT)

(72) Inventors: Giacomo Mandruzzato, Innsbruck (AT); Marek Polak, Innsbruck (AT)

(21) Appl. No.: 19/694,283

(22) Filed: Jun. 1, 2026

Related U.S. Application Data
(63) Continuation of application No. 18/288,155, filed on Apr. 28, 2022, now abandoned.
(60) Provisional application No. 63/181,801, filed on Apr. 29, 2021.

(51) Int. Cl. A61N 1/36; A61N 1/05

(52) U.S. Cl. CPC A61N 1/36039; A61N 1/0541

(57) ABSTRACT

Approaches are described for adjusting a cochlear implant system that has an electrode array with multiple electrode contacts, which is implanted in a patient. For selected individual electrode contacts, a corresponding channel-specific monoaural interaction component (MIC) score is calculated that represents a channel interaction factor based on a ratio of: i. an electrically evoked auditory brainstem response (eABR) measurement of an electrical stimulation signal applied to the individual electrode contact, and ii. a sum of individual eABR measurements from simultaneous electrical stimulation of selected electrode contacts nearest to the individual electrode contact. Each electrode contact having a channel-specific MIC score below a MIC score threshold value is then deactivated, whereby electrical stimulation signals are not delivered to deactivated electrode contacts.

### CROSS-REFERENCE TO RELATED APPLICATIONS

This application is a continuation of U.S. patent application Ser. No. 18/288,155, filed Oct. 24, 2023, which in turn is a U.S. national phase entry under 35 USC § 371 of Patent Cooperation Treaty Application No. PCT/US2022/026722 filed Apr. 28, 2022, which in turn claims priority from U.S. Provisional Application 63/181,801, filed Apr. 29, 2021. Each of the above-described applications is hereby incorporated herein by reference in their entirety.

### TECHNICAL FIELD

The present invention relates to hearing implants, and more specifically to fit customization in cochlear implant applications.

### BACKGROUND ART

A normal ear transmits sounds as shown in FIG. 1 through the outer ear 101 to the tympanic membrane (eardrum) 102, which moves the bones of the middle ear 103 (malleus, incus, and stapes) that vibrate the oval window and round window openings of the cochlea 104. The cochlea 104 is a long narrow duct wound spirally about its axis for approximately two and a half turns. It includes an upper channel known as the scala vestibuli and a lower channel known as the scala tympani, which are connected by the cochlear duct. The cochlea 104 forms an upright spiraling cone with a center called the modiolar where the spiral ganglion cells of the acoustic nerve 113 reside. In response to received sounds transmitted by the middle ear 103, the fluid-filled cochlea 104 functions as a transducer to generate electric pulses which are transmitted to the cochlear nerve 113, and ultimately to the brain.

Hearing is impaired when there are problems in the ability to transduce external sounds into meaningful action potentials along the neural substrate of the cochlea 104. To improve impaired hearing, auditory prostheses have been developed. For example, when the impairment is related to operation of the middle ear 103, a conventional hearing aid may be used to provide acoustic-mechanical stimulation to the auditory system in the form of amplified sound. Or when the impairment is associated with the cochlea 104, a cochlear implant with an implanted stimulation electrode can electrically stimulate auditory nerve tissue with small currents delivered by multiple electrode contacts distributed along the electrode.

FIG. 1 also shows some components of a typical cochlear implant system which includes an external microphone that provides an audio signal input to an external signal processor 111 where various signal processing schemes can be implemented. The processed signal is then converted into a digital data format, such as a sequence of data frames, for transmission into the implant processor 108 via headpiece 107. Besides receiving the processed audio information, the implant processor 108 also performs additional signal processing such as error correction, pulse formation, etc., and produces a stimulation pattern (based on the extracted audio information) that is sent through an electrode lead 109 to an implanted electrode array 110. Typically, this electrode array 110 includes multiple electrode contacts 112 on its surface that provide selective stimulation of the cochlea 104. Each electrode contact 112 provides stimulation signals for a specific defined band of audio frequencies, and in that context electrode contacts are also referred to as electrode channels.

On average, speech understanding outcomes in patients with cochlear implants are good and improve the quality of life. Still, important inter-individual variability can significantly affect the outcomes. Several factors affect this variability, among them electrical interactions between single channels inside the cochlea, which are caused by residual polarization and refractory effects (Julie A. Bierer and Leonid Litvak, “Reducing channel interaction through cochlear implant programming may improve speech perception: current focusing and channel deactivation,” Trends in hearing 20 (2016): 2331216516653389; incorporated herein by reference in its entirety). The “Continuous Interleaved Sampling (CIS)” stimulation strategy (Wilson et al., Better Speech Recognition With Cochlear Implants, Nature, vol. 352:236-238 (1991); incorporated herein by reference in its entirety) was intended to eliminate such channel interactioNn. In CIS, symmetrical biphasic current pulses are used which are strictly non-overlapping in time, and the stimulation rate per channel typically is higher than 800 pulses/sec.

eABR (electrically auditory brainstem response) is an auditory evoked potential elicited after an electrical stimulation from the cochlear electrode array. As seen in the example in FIG. 2, this is recordable in the 10 ms after stimulation using far-field surface recording electrodes placed on the scalp. The eABR response is originated from the auditory nerve (eII), the cochlear nucleus (eIII), and the inferior colliculus/lateral lemniscus (eV) of the brainstem. Wave eI is not present because it is masked by the electrical stimulus artifact in the first millisecond (equivalent to the eCAP recordable by the intracochlear contacts). The biggest and most clear peak is wave eV, and it is also often used in audiology for threshold estimation and latency analysis, similar to the acoustic counterpart wave V of the ABR. Both eABR and eCAP recordings can provide information about the electrophysiology of the cochlea and auditory nerve that can be useful for post-operative fitting of the implant to the patient.

Guevara, Nicolas, et al. “A cochlear implant performance prognostic test based on electrical field interactions evaluated by eABR (electrical auditory brainstem responses).” PloS one 11.5 (2016): e0155008 (incorporated herein by reference in its entirety) demonstrated that it is possible to forecast the patients' outcomes based on the channel interaction of eABR recording via standard far field recording on the scalp. Guevara describes calculating the wave V amplitude ratio of summed eABR amplitude recorded for stimulation of different single channels along the cochlea to an eABR recorded when simultaneously stimulating those channel. This ratio is used to estimate a channel interaction factor referred to as a monoaural interaction component (MIC). MIC score for each subject ultimately correlates with speech comprehension. When there is no channel interaction, the sum of the eABR amplitudes obtained by individual stimulations should be very similar to the eABR obtained with the multi-electrode stimulation, and therefore, the MIC score equals 1. When high channel interaction is present, the sum of the eABR amplitudes obtained from individual stimulations should then be N times larger than the eABR obtained from the multi-electrode stimulation, where N is the number of stimulating channels, and therefore the MIC score equals N.

Speech perception could be also improved by improving the quality of the electrode-neuron interface. Speech perception is improved by disabling channels with “poor” electrode-neuron interfaces. At least one recent study showed improvements in speech perception scores when a subset of electrodes was deactivated with the intent of improving a psychophysical percept or reducing channel interaction (Bierer and Litvak 2016). Channels were deactivated when the model suggested a high degree of overlapping stimulation patterns with neighboring channels. Until now, there have not been automatic methods able to detect channel interaction based on objective measures.

### SUMMARY

Embodiments of the present invention are directed to approaches for adjusting a cochlear implant system that has an electrode array with multiple electrode contacts, which is implanted in a patient. For selected individual electrode contacts, a corresponding channel-specific monoaural interaction component (MIC) score is calculated that represents a channel interaction factor based on a ratio of: i. an electrically evoked auditory brainstem response (eABR) measurement of an electrical stimulation signal applied to the individual electrode contact, and ii. a sum of individual eABR measurements from simultaneous electrical stimulation of selected electrode contacts nearest to the individual electrode contact. Each electrode contact having a channel-specific MIC score below a MIC score threshold value is then deactivated, whereby electrical stimulation signals are not delivered to deactivated electrode contacts.

In further specific embodiments, patient fitting map values are adjusted to account for any deactivated electrode contacts. Wave eV amplitude may be used in the eABR measurements. The selected electrode contacts nearest to the individual electrode contact may be the nearest two electrode contacts or the nearest four electrode contacts. The sum of individual eABR measurements from simultaneous electrical stimulation of selected electrode contacts nearest to the individual electrode contact may include or exclude an individual eABR measurement of the individual electrode contact. The selected electrode contacts may include all of the electrode contacts in the electrode array. Calculating channel-specific monoaural interaction component (MIC) scores may include normalizing the MIC scores to a range [0, 1]. The MIC score threshold value may be a user-selectable value.

Embodiments also include a cochlear implant adjustment system using a method according to any of the above, and a computer program product implemented in a tangible computer readable storage medium for adjusting an implanted electrode array of a cochlear implant to an implanted patient that includes program code for performing a method according to any of the above.

### BRIEF DESCRIPTION OF THE DRAWINGS

FIG. 1 shows anatomical structures in a human ear having a cochlear implant system.

FIG. 2 shows an example of eABR waveform recordings elicited after an electrical stimulation from a cochlear electrode array.

FIG. 3 shows a block diagram of a cochlear implant adjustment system according to one specific embodiment of the present invention.

FIG. 4 shows various logical steps in performing a cochlear implant adjustment process according to one specific embodiment of the present invention.

FIG. 5 shows a display of electrode channel MIC scores as used in patient fitting software according to an embodiment of the present invention.

FIG. 6 shows a display of electrode channel MIC scores as used in patient fitting software according to another embodiment of the present invention.

FIG. 7 shows a display of electrode channel MIC scores as used in patient fitting software according to another embodiment of the present invention.

FIG. 8 shows a display of electrode channel MIC scores as used in patient fitting software according to another embodiment of the present invention.

FIG. 9 shows an exemplary display of measured electrode channel MIC scores that can be used in patient fitting software according to another embodiment of the present invention.

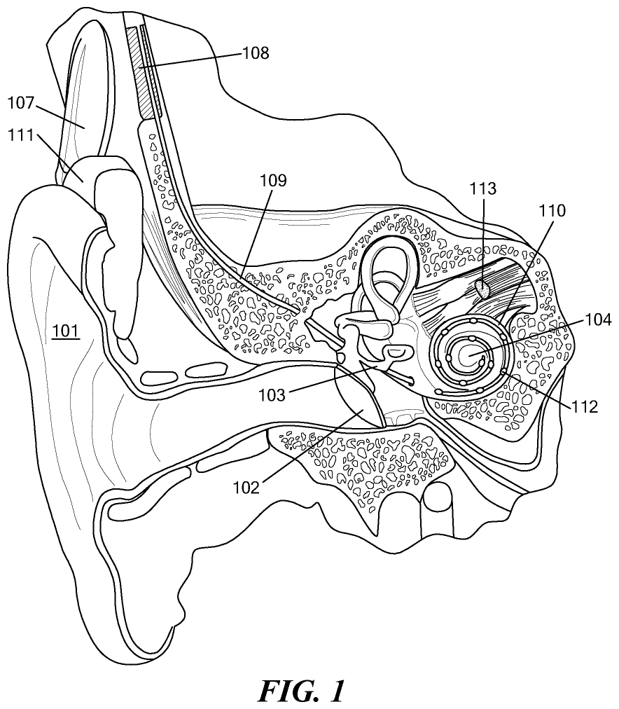

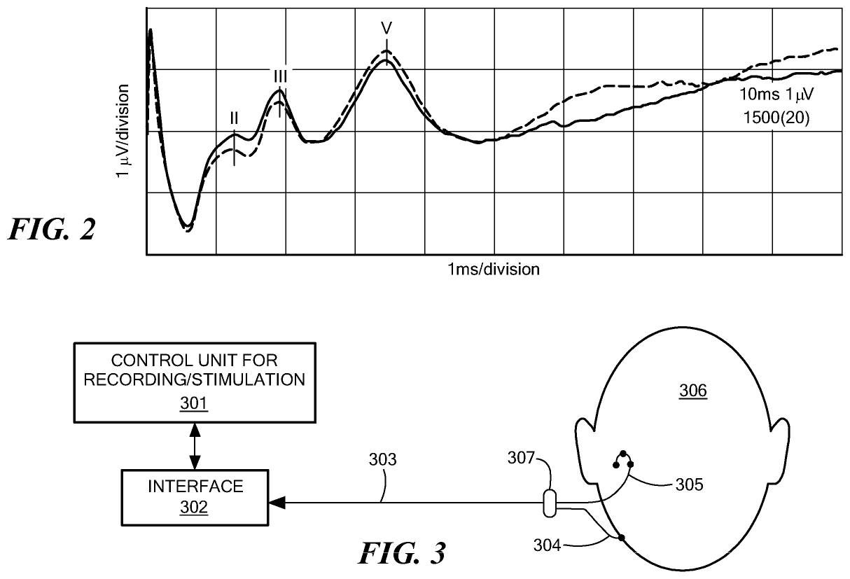

### DETAILED DESCRIPTION

Embodiments of the present invention such as those discussed within can give an objective indication of an electrode channel with excessive channel interaction. More specifically, embodiments of the present invention calculate channel-specific MIC scores as described below, and then switch off electrode contacts based on the MIC value and adjust fitting map values such as MCL and THR.

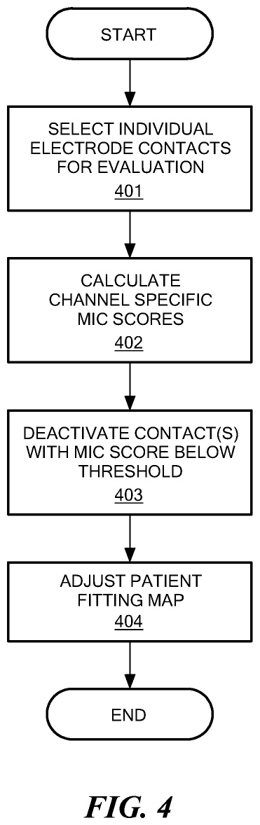

FIG. 3 shows a block diagram of a cochlear implant adjustment system and FIG. 4 shows various logical steps in performing a cochlear implant adjustment process according to one specific embodiment of the present invention. Control Unit 301 for Recording and Stimulation, for example, a Med-El Maestro CI system, includes at least one hardware implemented processor and a computer program product implemented in a tangible computer readable storage medium configured for generating electrical stimulation signals and analyzing response measurements such as eABR waveforms. Connected to the Control Unit 301 is an Interface Box 302, for example, a Diagnostic Interface System such as the MAX Programming Interface conventionally used with the Maestro CI system that formats and distributes the input and output signals between the Control Unit 301 and the system components implanted in the Patient 306. For example, as shown in FIG. 3, there may be an Interface Lead 303 connected at one end to the Interface Box 302 and at the other end having Electrode Plug 307 that then divides into a Cochlear Implant Electrode 304 and an Extra-Cochlear Ground Electrode 305. It should be noted that other methods and means to interconnect equally work without limitation, for example Interface Lead 303 may be a wireless connection, where e.g. such a wireless connection may communicative (and/or transcutaneously) couple Interface Box 302 with Cochlear Implant Electrode 304 and an Extra-Cochlear Ground Electrode 305 when implanted in the patient body. After or during delivering a stimulation pulse, a Cochlear Implant Electrode 304 may be used as a sensing element to determine current and voltage characteristics of the adjacent tissue, for example, for use in measuring eABRs.

Using an adjustment system such as the one depicted in FIG. 3, an adjustment process according to an embodiment of the present invention starts by selecting a group of individual electrode contacts for evaluation, step 401. Then channel-specific monoaural interaction component (MIC) scores are calculated for the selected electrode contacts, step 402, that represent a channel interaction factor based on a ratio of: i. an electrically evoked auditory brainstem response (eABR) measurement of an electrical stimulation signal applied to the individual electrode contact, and ii. a sum of individual eABR measurements from simultaneous electrical stimulation of selected electrode contacts nearest to the individual electrode contact. Calculating channel-specific monoaural interaction component (MIC) scores may include normalizing the MIC scores to a range [0, 1].

Each electrode contact having a channel-specific MIC score below a MIC score threshold value is then deactivated, step 403, whereby electrical stimulation signals are not delivered to deactivated electrode contacts. The MIC score threshold value may be a user-selectable value. In further specific embodiments, patient fitting map values are adjusted to account for any deactivated electrode contacts, step 404.

The eABR recordings may be referenced to a common ground on an exterior surface of the implant housing, or referenced to another electrode contact in the implanted electrode array. In an eABR waveform (with a reference electrode on the mastoid as a surface electrode, or on the implant housing for an intra-cochlear electrode contact), wave eV is the positive peak with a latency of around 3.8-4 msec. Wave eV amplitude is calculated from that peak to the previous or following valley, and this amplitude may be used in the eABR measurements. Since this peak amplitude is variable among subjects, peak amplitude may be also calculated as ratio of amplitudes of two peaks (normalized amplitude). For example, the amplitude of wave eV can be divided by the amplitude of wave eI or eIII.

The selected electrode contacts nearest to the individual electrode contact for the MIC calculation may be the nearest two electrode contacts or the nearest four electrode contacts. Or the selected electrode contacts may include all of the electrode contacts in the electrode array. And the sum of individual eABR measurements from simultaneous electrical stimulation of selected electrode contacts nearest to the individual electrode contact may include or exclude an individual eABR measurement of the individual electrode contact. Several specific algorithms are now described in greater detail.

A type 1 MIC score approach uses MIC scores for all of the electrode contacts of the full electrode array, and channels with high interaction may be selected for deactivation. The type 1 MIC score calculates the channel interaction of each single channel (each electrode contact) by stimulating a group of selected electrode contacts (typically 3-4) and evaluating the channel interaction at different frequency codings, for example, within the apical-mid-basal part of the electrode array. This provides an understanding of the channel interactions for different cochlear areas and at different frequencies. Electrode channels with high interaction may not benefit the implanted user in term of speech recognition, and therefore adjustment of the fit MAP may be needed.

For some number NS of neighbouring electrode channels, for example NS=3, the selected electrode contacts are stimulated and a MIC score is calculated. For a specific channel i in the group NS, MICᵢ=the individual eABR elicited by stimulation on channel i/summed eABR elicited by simultaneous stimulation of channels i−1, i, i+1. This produces a MIC score in the range of [1, NS]. The closer the MIC score is to 1, the lower is the channel interaction. A decision threshold between low and high channel interaction can be selected, either a fixed value, or a user-selected value based on personal experience or based on the overall values of the MIC in the patient. For example, the threshold can be defined as the mean MIC+2 STD. Then if MICᵢ<MICthr, channel I can be deactivated.

Considering channel i, the selected neighbouring channels typically would be the two most adjacent channels: i+1 and i−1. Alternatively, an MCIᵢ formula can be also calculated extending the number of NS to 5 (considering therefore i+2 and i−2), or more. To calculate a MIC for the most apical (or most basal) channel, the channel i+1 and i+2 (or i−1 and i−2) channel may be used instead. MIC score can be also calculated using two adjacent electrodes (one for stimulation and one for recording) spanning all the cochlea. This would give an idea of the channel interaction in a precise electrode location.

In a second type 2 algorithm, the MIC score can be used to select an electrode channel to deactivate to improve speech perception. Individual channels are deactivated consecutively one after the another, recording the eABR and calculating the MIC score using all the other channels then active. At the end, for an electrode array with N electrode channels, N MIC scores are obtained. Those channels giving lower MIC scores with their deactivation are likely to generate channel interaction when they are activated. This suggests which channels to permanently deactivate for lower final channel interaction.

The algorithm can be summarized:

For a given channel i:
Record the eABR stimulating single channel i;
Switch off channel i;
Record a summation eABR simultaneously stimulating multiple channels, except channel i:
Calculate MIC score which represents the results of channel interaction of channel i: MICᵢ=eABR elicited stimulating on channel i/summed eABR elicited by simultaneous stimulation of channels (1, . . . i−1, i+1, . . . . N); and
At the end, suggestion to deactivate channel(s) which give higher MIC score:
   If MICᵢ>MICₜₕᵣ:
      Deactivate channel i (For MIC threshold decision, see before).

For N electrode channels, the number of EABR recordings would be: N (1+1)=2N. For increased precision, the algorithm can be used to focus only on a selected region of the cochlea that codes for a given selected acoustic frequency (thereby focusing only on a narrow sub-group of auditory nerve fibers), only a subset (NS) of neighbouring stimulating channels eliciting eABR can be used (e.g, 3). For NS=3 and N electrode channels, the number of eABR recordings would be:

$${N}_{eABR} = N + ( N - ( NS - 1 ) ) = N + ( N - 2 ) = 2 ( N - 1 ) .$$

The advantage of this type 2 procedure is that the neighbouring MIC scores do separate channels and the MICᵢ score informs on the channel interaction for all neighbouring channels (i−1, i, i+1).

In a third type 3 alternative, channel interaction can be identified by adding (switching-on) only one selected electrode channel to the current MAP configuration which was previously deactivated. The activated channel with a higher MIC score would add more channel interactions with its activation, which suggests whether the activation or the deactivation of a single electrode channel will reduce the channel interaction. This is useful if the user has already a satisfactory MAP on most of the electrode channels, and the fitting is not satisfactory on just one or a few channels, suggesting that on those channels there is a possibility of channel interaction. To calculate MIC score only adding a single channel, MICᵢ=(eABR stimulating multiple active channels except channel i+eABR stimulating channel i)/eABR stimulating multiple channels simultaneously. This type 3 algorithm needs only 3 eABR recordings to check channel interaction on one channel. Using this formula, the MIC score will be in the range of [1, n], where the closer the score is to 1, the lower is the channel interaction.

Definition of multiple stimulation. Multiple simultaneous stimulation refers to stimulation of more than one electrode channel. For calculation of the MIC score, multiple stimulation may include all the electrode channels of the electrode array (from 1 to N). Multiple stimulation can also refer to a subgroup of neighbouring electrode channels; for example, 1, 2, . . . i, where 2<i≤N, or, i, i+1, . . . . N, where 0<i≤N.

Stimulation Amplitude. The stimulation amplitude has to be enough to elicit an eABR waveform response. But if the stimulation amplitude is too high, it may create discomfort to the implant user such as side effect myogenic stimulation (e.g., facial nerve). Every subject has a different stimulation threshold (THR) and most comfortable level (MCL), which can also vary within the same patient on different electrode channels. Also, for calculating MIC scores, the eABR is elicited stimulating both individual electrode channels and also simultaneously stimulating multiple electrode channels. These two different stimulation patterns can have different THR and different MCL levels. The stimulation amplitude to elicit eABR for the MIC score calculation therefore can vary accordingly. Stimulation amplitude can be derived from the fitting MAP, taking the THR level, MCL level, or a certain percentage of the MCL level (for example, 75% of MCL level) for each electrode channel. Alternatively, subjective testing can help to determine the THR and MCL level.

Normalization. The MIC scores can be normalized based on the number of electrode channels used in order to compare different channel MIC scores. The specific normalization calculation used can vary depending on the specific type of MIC score calculation. For example, for type 1 and type 3 MIC scores, for 3 electrode channels, would span a range value from 1 (no channel interaction) to 3, while on 5 electrode channels, the MIC score would span a range value from 1 (no channel interaction) to 5. A normalization formula is:

$$T ( x , y ) = ( x \frac{1}{NS} - \frac{1}{NS} , y )$$

where NS is the number of neighbouring electrode channels. After normalization, the MIC score would span a range from 0 (no channel interaction) to 1, independently from the number of channels used.

With type 2 MIC score, the MIC score on 3 electrode channels would span a range value from ½ (no channel interaction) to 1, while the MIC score on 4 electrode channels would span a range value from ⅓ (no channel interaction) to 1. In that case, the general transformation/normalization would be:

$$T ( x , y ) = ( x ( \frac{NS}{NS - 1} ) - \frac{1}{NS - 1} , y )$$

where NS is the number of neighbouring electrode channels used to calculate the MIC score. After normalization, the MIC score would span a range from 0 (no channel interaction) to 1, independently from the number of channels used.

It was previously explained that the wave V of the eABR recorded by scalp electrode (far field) provides good results in MIC calculation. This approach excludes the use of ECAP recording for various reasons.

First, the electrical artefact generated on the simultaneous stimulation is too large, covering the neural response. eCAP is characterized by a single negative peak (N1) in the latency range of 0.2 to 0.4 msec, where the electrical artefact also is present (e.g., Miller, Charles A., Paul J. Abbas, and Barbara K. Robinson. “The use of long-duration current pulses to assess nerve survival.” Hearing research 78.1 (1994): 11-26; incorporated herein by reference in its entirety). eABR peaks come later in time, having a latency for eIII of about 1.9 ms and eV of about 3.9 ms (e.g., Hodges, Annelle V., et al. “Electric auditory brain-stem responses in Nucleus multichannel cochlear implant users.” Archives of Otolaryngology-Head & Neck Surgery 120.10 (1994): 1093-1099; incorporated herein by reference in its entirety). In the expected latency of eABR, the electrical artefact is already vanished. Experience teaches us that even using electrical artefact reduction algorithm for eCAP would not be satisfactory with simultaneous stimulation.

In addition, the ECAP response does not take in account the integration process from different cochlear areas. Embodiments of the present make use of the wave V of eABR, which is a response from the brainstem where some elaboration of the auditory information takes place. By contrast, ECAP recordings, being near field evoked potentials, are a post-stimulation response of the spiral ganglion cells of the distal portion of the auditory nerve. Furthermore, ECAPs from simultaneous stimulation of multiple electrode channels would be the sum of different independent groups of nerve fibres. Variability in the electrode-neuron interface in the cochlea also plays a role in the ECAP recording, while eABR, being a response from the auditory nerve and the brainstem, is more robust in that sense.

The foregoing describes calculating the eV amplitude and using that for the MIC score calculation. But other eABR waveform peaks could also be used, for example, wave ell and/or wave eIII. Wave eI is masked by the electrical stimulus artefact and therefore is not visible or usable. In addition, the MIC score can be extended also to other auditory evoked responses, for example middle latency (eMLR) and late latency (cortical) potentials (eLLR or CAEP). These elicited evoked potentials are from part of the brainstem and thalamus up to the cortical areas (primary and secondary auditory cortex). Therefore, at that stage, more processing of sound integration takes place, for example general sound detection, pitch and level detection. eMLR is characterized by a negative peak (Na) at approximately 15-18 msec, positive peak (Pa) at approximately 25-30 msec, and negative peak (Nb) at about 30-40 msec (Firszt, J. B., Chambers, R. D., Kraus, N. and Reeder, R. M., 2002. Neurophysiology of cochlear implant users I: effects of stimulus current level and electrode site on the electrical ABR, MLR, and N1-P2 response. Ear and hearing, 23 (6), pp. 502-515; incorporated herein by reference in its entirety). eLLR or CAEP is characterized by the complex P1-N1-P2 with positive and negative peaks, having presence and latency strongly dependent from the development of the auditory pathway. In adults, N1 has latency approximately 80 to 110 ms and P2 at approximately 160 to 210 ms (Näätänen, R. and Picton, T., 1987. The N1 wave of the human electric and magnetic response to sound: a review and an analysis of the component structure. Psychophysiology, 24 (4), pp. 375-425; incorporated herein by reference in its entirety). Analysis of these responses in combination with stimulation pattern and MIC calculation as above would provide useful information about channel interaction for improving the fitting MAP.

For recording the eABR waveforms, one electrode contact adjacent to the stimulating contact can be used as a recording electrode, for example, el+1. Some embodiments could use multiple recording electrodes, for example, el+1, el-1, . . . and then increasing the recording time. Any recording electrode(s) have to not be stimulating and so cannot be used when summing eABRs from simultaneous stimulation of multiple channels.

Since eABR is the response of the brainstem, that response may not change in terms of morphology, amplitude and latency when changing the recording electrode along the cochlea. This is due to the physical context that the electrode array is coiled within the cochlea spanning a radius of a few millimeters, while the brainstem is a bigger structure that is situated further away toward the center of the body. Therefore, evoked potentials recorded from the intracochlear electrodes would be very similar among the individual electrode channels. Moreover, since the recording is referenced to an external ground or to another contact outside the cochlea, the difference in electrical potential between different intra-cochlear electrode contacts is even more attenuated.

The eABR signal can be also normalize to the electrode impedance (IFT). The higher the electrode impedance is (which is the result of tissue impedance around the electrode contact), the lower is the amplitude. Normalizing the eABR signal to the electrode impedance allows a better comparison of eABR recordings from different electrode contacts, especially in calculating the MIC score where eABRs of different electrode channels have to evaluated and summed together.

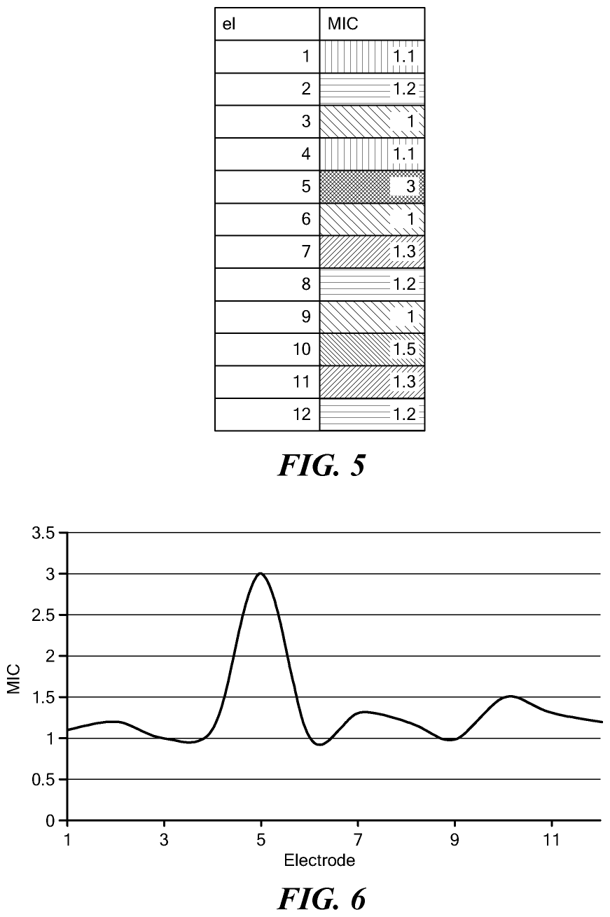

The calculated MIC scores are then made available to the doctor/audiologist. The MIC score can be visualized on the clinical fitting software in a simple and intuitive way, so that clinicians can easily recognize the channel interaction. For example, as shown in FIG. 5, the MIC score value can displayed for each electrode channel (with coded line shading). In FIG. 5, the darker line shading used in channel 5 indicates high channel interaction (e.g., above the channel interaction threshold), while the other shading in the other electrode channels shows lower channel interaction.

Alternatively, the MIC scores can be plotted on an x-y plot as shown in FIG. 6, where the x-axis is the number of the electrode channel and the y-axis the corresponding MIC score. This plot shown how the MIC score change along the cochlea from the apical end to the basal end.

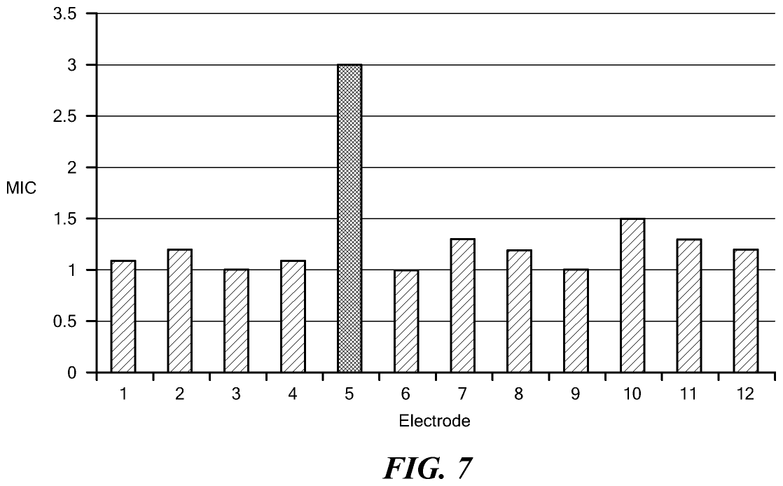

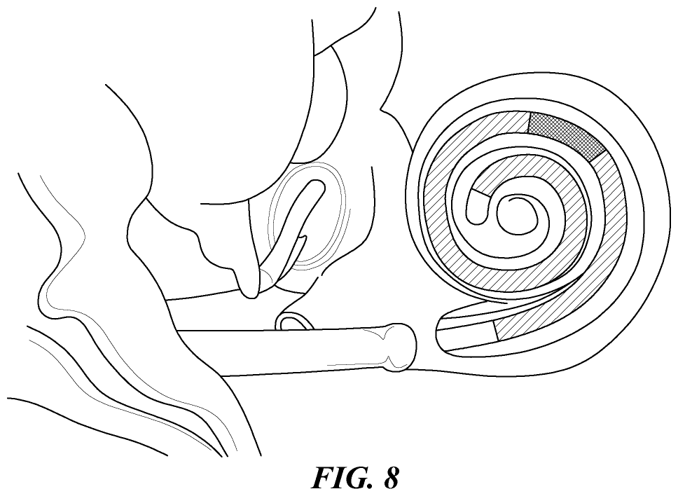

FIG. 7 depicts the MIC scores using a bar plot. FIG. 8 depicts an intuitive display of the MIC scores in an image of the cochlea with an inserted electrode array were regions with high channel interaction are highlighted in dark line shading.

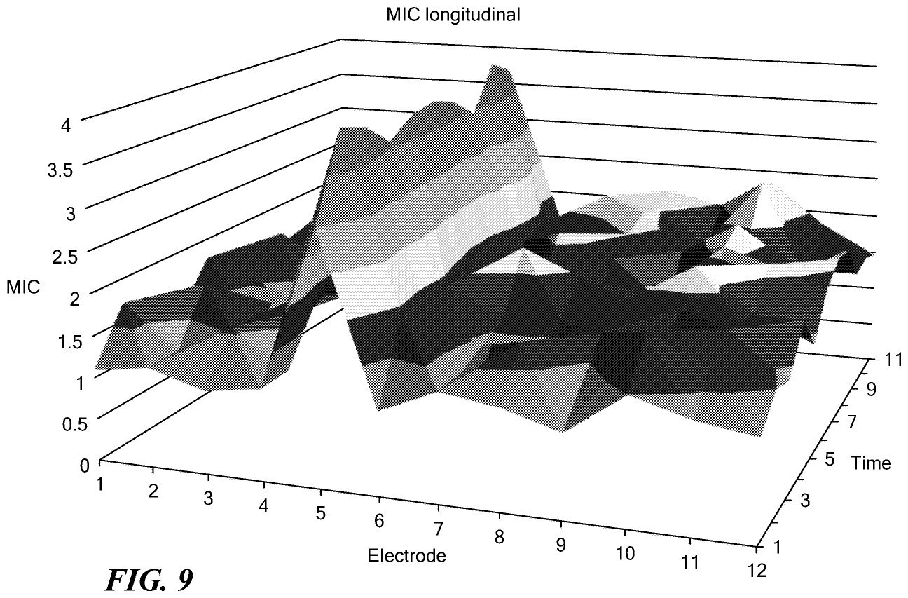

If a patient reports speech degradation, possible causes can include nerve degeneration, change of conductivity of cochlea tissue, etc. These changes can also be reflected in channel interaction and therefore indicated by the MIC score. Comparing MIC scores at different time points may help the audiologist to objectively determine the phenomena, find the causes of speech degradation, and react promptly deactivating channels with high interaction. Thus the fitting display can reflect the recording of MIC scores at different time points as shown in FIG. 9. On the x-axis the number of the electrode channel is shown, on the y-axis the MIC score corresponding to each electrode channel, and on the z-axis the array of MIC scores for each time point. Alternatively, a colored numeric matrix representation also can be used with color coding as described above.

The MIC scores are intended as suggestion to the audiologist/doctor with respect to deriving information for the fitting map, for adjusting MCL and THR to minimize the saturation and channel interaction, and estimating possible benefits of the cochlear implant and future possible outcomes for a given patient.

Embodiments of the invention may be implemented in part in any conventional computer programming language. For example, preferred embodiments may be implemented in a procedural programming language (e.g., “C”) or an object oriented programming language (e.g., “C++”, Python). Alternative embodiments of the invention may be implemented as pre-programmed hardware elements, other related components, or as a combination of hardware and software components.

For example, a pseudo code representation of a generic embodiment might be set forth as follows:

```
Process MIC_Score
  select individual electrode contacts for evaluation
  calculate channel-specific MIC scores
  deactivate electrode contacts below threshold
  adjust patient fitting MAP
```

Embodiments can be implemented in part as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

Although various exemplary embodiments of the invention have been disclosed, it should be apparent to those skilled in the art that various changes and modifications can be made which will achieve some of the advantages of the invention without departing from the true scope of the invention.

What is claimed is:

1. A cochlear implant adjustment system for adjusting a cochlear implant system having an electrode array with a plurality of electrode contacts implanted in a patient, the system comprising:
means for calculating for selected individual electrode contacts a corresponding channel-specific monoaural interaction component (MIC) score representing a channel interaction factor based on a ratio of:
i. an electrically evoked auditory brainstem response (eABR) measurement of an electrical stimulation signal applied to the individual electrode contact, and
ii. a sum of individual eABR measurements from simultaneous electrical stimulation of a selected plurality of electrode contacts nearest to the individual electrode contact; and
means for deactivating each electrode contact having a channel-specific MIC score below a MIC score threshold value, whereby electrical stimulation signals are not delivered to deactivated electrode contacts.

2. The system according to claim 1, further comprising means for adjusting patient fitting map values to account for any deactivated electrode contacts.

3. The system according to claim 1, wherein wave eV amplitude is used in the eABR measurements.

4. The system according to claim 1, wherein the selected plurality of electrode contacts nearest to the individual electrode contact comprises the nearest two electrode contacts.

5. The system according to claim 1, wherein the selected plurality of electrode contacts nearest to the individual electrode contact comprises the nearest four electrode contacts.

6. The system according to claim 1, wherein the sum of individual eABR measurements from simultaneous electrical stimulation of the selected plurality of electrode contacts nearest to the individual electrode contact includes an individual eABR measurement of the individual electrode contact.

7. The system according to claim 6, wherein the selected plurality of electrode contacts includes all of the plurality of electrode contacts in the electrode array.

8. The system according to claim 1, wherein the sum of individual eABR measurements from simultaneous electrical stimulation of the selected plurality of electrode contacts nearest to the individual electrode contact excludes an individual eABR measurement of the individual electrode contact.

9. The system according to claim 1, wherein calculating channel-specific monoaural interaction component (MIC) scores includes normalizing the MIC scores to a range [0, 1].

10. The system according to claim 1, wherein the MIC score threshold value is a user-selectable value.

11. A computer program product implemented in a tangible computer readable storage medium for adjusting a cochlear implant system having an electrode array with a plurality of electrode contacts implanted in a patient, the product comprising:
program code for calculating for selected individual electrode contacts a corresponding channel-specific program code for monoaural interaction component (MIC) score representing a channel interaction factor based on a ratio of:
i. an electrically evoked auditory brainstem response (eABR) measurement of an electrical stimulation signal applied to the individual electrode contact, and
ii. a sum of individual eABR measurements from simultaneous electrical stimulation of a selected plurality of electrode contacts nearest to the individual electrode contact; and
program code for deactivating each electrode contact having a channel-specific MIC score below a MIC score threshold value, whereby electrical stimulation signals are not delivered to deactivated electrode contacts.

12. The product according to claim 11, further comprising program code for adjusting patient fitting map values to account for any deactivated electrode contacts.

13. The product according to claim 11, wherein wave eV amplitude is used in the eABR measurements.

14. The product according to claim 11, wherein the selected plurality of electrode contacts nearest to the individual electrode contact comprises the nearest two electrode contacts.

15. The product according to claim 11, wherein the selected plurality of electrode contacts nearest to the individual electrode contact comprises the nearest four electrode contacts.

16. The product according to claim 11, wherein the sum of individual eABR measurements from simultaneous electrical stimulation of the selected plurality of electrode contacts nearest to the individual electrode contact includes an individual eABR measurement of the individual electrode contact.

17. The product according to claim 16, wherein the selected plurality of electrode contacts includes all of the plurality of electrode contacts in the electrode array.

18. The product according to claim 11, wherein the sum of individual eABR measurements from simultaneous electrical stimulation of the selected plurality of electrode contacts nearest to the individual electrode contact excludes an individual eABR measurement of the individual electrode contact.

19. The product according to claim 11, wherein calculating channel-specific monoaural interaction component (MIC) scores includes normalizing the MIC scores to a range [0, 1].

20. The product according to claim 11, wherein the MIC score threshold value is a user-selectable value.

21. A computer based method implemented using at least one hardware implemented processor for adjusting a cochlear implant system having an electrode array with a plurality of electrode contacts implanted in a patient, the method comprising:
using the at least one hardware implemented processor to perform the steps of:
calculating for selected individual electrode contacts a corresponding channel-specific monoaural interaction component (MIC) score representing a channel interaction factor based on a ratio of:
an electrically evoked auditory brainstem response (eABR) measurement of an electrical stimulation signal applied to the individual electrode contact, and
a sum of individual eABR measurements from simultaneous electrical stimulation of a selected plurality of electrode contacts nearest to the individual electrode contact; and
deactivating each electrode contact having a channel-specific MIC score below a MIC score threshold value, whereby electrical stimulation signals are not delivered to deactivated electrode contacts.

22. The method according to claim 21, further comprising adjusting patient fitting map values to account for any deactivated electrode contacts.

23. The method according to claim 21, wherein wave eV amplitude is used in the eABR measurements.

24. The method according to claim 21, wherein the selected plurality of electrode contacts nearest to the individual electrode contact comprises the nearest two electrode contacts.

25. The method according to claim 21, wherein the selected plurality of electrode contacts nearest to the individual electrode contact comprises the nearest four electrode contacts.

26. The method according to claim 21, wherein the sum of individual eABR measurements from simultaneous electrical stimulation of the selected plurality of electrode contacts nearest to the individual electrode contact includes an individual eABR measurement of the individual electrode contact.

27. The method according to claim 26, wherein the selected plurality of electrode contacts includes all of the plurality of electrode contacts in the electrode array.

28. The method according to claim 21, wherein the sum of individual eABR measurements from simultaneous electrical stimulation of the selected plurality of electrode contacts nearest to the individual electrode contact excludes an individual eABR measurement of the individual electrode contact.

29. The method according to claim 21, wherein calculating channel-specific monoaural interaction component (MIC) scores includes normalizing the MIC scores to a range [0, 1].

30. The method according to claim 21, wherein the MIC score threshold value is a user-selectable value.
